(19) United States
(12) Patent Application Publication
(10) Pub. No.: US 2026/0224508 A1
(43) Pub. Date: Aug. 6, 2026

(54) Multilayered Therapeutic Patch Structure for Local Oral Administration

(71) Applicant: Maoren Li, Sacramento, CA (US)

(72) Inventor: Maoren Li, Sacramento, CA (US)

(21) Appl. No.: 19/555,140

(22) Filed: Mar. 3, 2026

(51) Int. Cl. A61K 9/70; A61K 9/00; A61K 45/06

(52) U.S. Cl. CPC A61K 9/7084; A61K 9/006; A61K 45/06

(57) ABSTRACT

A multilayered toothache patch for targeted local oral administration comprises: (1) a base support and isolation layer of waterproof or semi-permeable medical-grade polymer forming the outer surface, preventing saliva infiltration and blocking drug leakage to non-target tissue; (2) a central active treatment zone of porous biocompatible carrier material loaded with therapeutic active ingredients for treating tooth pain by non-anesthetic mechanisms, having a grid-textured surface to optimize drug loading and sustained release; and (3) at least two side anchoring and blocking zones symmetrically flanking the central zone, providing adhesion to moist gingival or mucosal tissue and acting as physical barriers preventing lateral migration of dissolved drug substance. Together, the three zones achieve targeted, unidirectional drug delivery confined to the affected site, offering a non-invasive alternative to systemic analgesics and invasive dental procedures.

### FIELD OF THE INVENTION

The present invention relates to the technical field of oral medical devices and drug delivery systems. More particularly, the present invention relates to a multilayered toothache patch structure configured for site-specific local administration to oral mucosa and periodontal tissue, for the targeted delivery of active therapeutic ingredients to treat tooth pain and associated periodontal conditions without reliance on nerve anesthesia, with the objective of achieving targeted precision therapy.

### BACKGROUND OF THE INVENTION

Toothache is one of the most common and debilitating forms of acute pain experienced by individuals worldwide. The causes are varied, including dental caries, pulpitis, periodontitis, pericoronitis, and other inflammatory or infectious conditions affecting the teeth, gums, and surrounding oral tissues.

Conventional approaches to tooth pain management present significant limitations. Systemic oral analgesics, while widely used, must traverse the bloodstream before reaching the affected site, resulting in delayed onset of action and systemic side effects including gastrointestinal disturbance, hepatotoxicity, and cardiovascular effects. Topical anesthetic gels provide localized surface relief but do not penetrate to the deeper tissue layers where pathological processes originate, offering only symptomatic relief of limited duration.

For more severe or chronic dental pain, clinical practice often involves irreversible, invasive interventions such as tooth extraction, root canal therapy, and subsequent restoration procedures including dental implants. These approaches involve significant patient discomfort, prolonged recovery, substantial financial cost, and permanent alteration of natural dental anatomy.

Furthermore, existing mucosal drug delivery systems in the oral cavity suffer from challenges including inadequate adhesion to moist mucosal surfaces, rapid dilution and washout by saliva, uncontrolled drug diffusion to non-target areas, and difficulty maintaining therapeutic drug concentrations at the site of action for sufficient duration.

There exists, therefore, an unmet clinical need for a non-invasive, site-specific oral drug delivery device that provides prolonged, targeted delivery of therapeutic agents directly to affected oral tissue, overcomes the challenges of the moist oral environment, and confines drug action to the lesion area to maximize therapeutic efficacy while minimizing systemic drug exposure.

The present invention addresses these deficiencies by providing a multilayered toothache patch structure with a novel combination of a base isolation layer, a central active treatment zone, and flanking side anchoring and blocking zones that collectively ensure stable fixation, unidirectional drug delivery, and effective prevention of drug leakage.

### SUMMARY OF THE INVENTION

In one aspect, the present invention provides a multilayered toothache patch structure for targeted local oral administration, comprising: (a) a base support and isolation layer forming the outermost surface of the patch; (b) a central active treatment zone disposed on a first face of the base support and isolation layer; and (c) at least two side anchoring and blocking zones disposed on the first face of the base support and isolation layer, symmetrically flanking the central active treatment zone along the longitudinal edges thereof.

In certain embodiments, the base support and isolation layer is formed from a medical-grade waterproof or semi-permeable polymeric material configured to substantially prevent permeation of saliva into the patch from the oral side and to substantially prevent release of active ingredients from the outer surface of the patch.

In certain embodiments, the central active treatment zone comprises a porous, highly adsorptive, biocompatible carrier material loaded with one or more active therapeutic ingredients effective to treat or alleviate tooth pain by mechanisms other than nerve anesthesia.

In certain embodiments, the side anchoring and blocking zones are configured to provide adhesion to moist gingival or mucosal tissue and to act as a physical barrier preventing lateral migration of liquefied or dissolved drug substances from the central active treatment zone.

It is a primary object of the present invention to provide a targeted, non-invasive oral drug delivery system for the treatment of tooth pain.

It is a further object to provide a patch structure that maintains stable fixation on moist oral mucosal and gingival surfaces during the period of drug delivery.

It is a further object to provide a unidirectional drug delivery mechanism that confines active ingredient penetration to the targeted tissue while preventing drug leakage to surrounding non-target areas.

### BRIEF DESCRIPTION OF THE DRAWINGS

The accompanying drawings, which are incorporated herein and form part of the specification, illustrate exemplary embodiments of the present invention and, together with the description, serve to explain the principles of the invention.

FIG. 1 is a composite view of the multilayered toothache patch structure according to an embodiment of the present invention, showing a top plan view, a side elevation view, and a perspective view, illustrating the relative arrangement and dimensions of the base support and isolation layer, the central active treatment zone, and the side anchoring and blocking zones.

FIG. 2 is a schematic diagram illustrating the structural layers and functional zones of the multilayered toothache patch according to an embodiment of the present invention.

### DETAILED DESCRIPTION OF THE PREFERRED EMBODIMENTS

The following detailed description is presented to enable any person skilled in the art to make and use the invention. For purposes of explanation, specific details are set forth in the following description to provide a thorough understanding of the present invention. However, it will be apparent to one skilled in the art that these specific details are not required to practice the invention. Descriptions of specific applications and methods are provided only as examples. Various modifications to the preferred embodiments will be readily apparent to those skilled in the art, and the general principles defined herein may be applied to other embodiments and applications without departing from the spirit and scope of the invention.

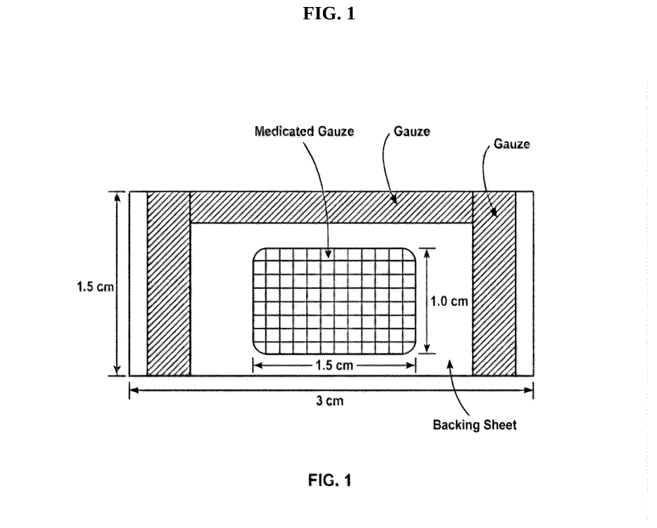

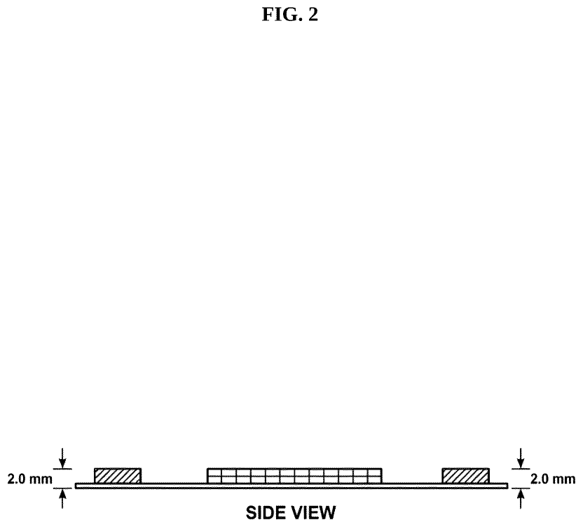

Referring now to FIG. 1 and FIG. 2, in a preferred embodiment, the present invention proposes an oral patch having a composite layered structure. From the overall appearance, the patch is substantially rectangular in plan view. The cross-section, as shown in the side view of FIG. 1, reveals a laminated structure of non-uniform thickness. The patch structure is composed of three primary functional zones: a continuous base support and isolation layer and multiple functional blocks arranged on the same face of the base support and isolation layer.

### A. Base Support and Isolation Layer (Backing Sheet)

The base support and isolation layer is located on the outermost layer of the patch, specifically on the side of the patch facing away from the oral mucosa during use. In the illustrated embodiment, the base support and isolation layer is a continuous, substantially planar film having outer dimensions of approximately 3.0 cm in length by 1.5 cm in width. As depicted in the side view, the thickness of the base support and isolation layer is substantially less than the thickness of the functional blocks disposed upon it, functioning as a flexible substrate sheet.

The base support and isolation layer serves as the structural foundation of the entire patch and functions as a physical barrier layer. It is preferably fabricated from a medical-grade waterproof or semi-permeable polymeric material. The base support and isolation layer performs the following technical functions: (i) blocking the rinsing and excessive dilution of drug components within the patch by saliva in the oral cavity, thereby ensuring sustained therapeutic efficacy; and (ii) preventing leakage of active ingredients to non-target areas such as the oral side wall mucosa, thereby achieving unidirectional, localized administration of the therapeutic agent.

Suitable materials for the base support and isolation layer include, but are not limited to, polyethylene film, polyurethane film, ethylene vinyl acetate copolymer film, polylactic acid film, and other biocompatible, flexible polymeric films possessing the requisite moisture barrier properties. The base support and isolation layer may optionally incorporate a semi-permeable membrane to permit limited moisture vapor transmission while restricting drug permeation therethrough.

### B. Central Active Treatment Zone (Medicated Gauze—Core Component)

The central active treatment zone is positioned centrally over the geometric center area of the base support and isolation layer. In a preferred embodiment, the planar dimensions of the central active treatment zone are approximately 1.5 cm in length by 1.0 cm in width. The side view of FIG. 1 shows that the central active treatment zone possesses a substantially greater thickness relative to the base support and isolation layer, approximately 2.0 mm, thereby forming a raised, three-dimensional drug reservoir pad structure.

In the top view of FIG. 1, the surface of the central active treatment zone is shown to exhibit a grid pattern or latticed texture. This surface texture indicates the employment of a porous weaving process or microstructure surface treatment, which increases specific surface area, enhances the loading capacity of the active ingredients, and optimizes the rate of drug release upon contact with the moist oral environment.

The central active treatment zone is the core functional element of the patch and is composed of a highly adsorptive, biocompatible carrier material including, but not limited to, modified gauze, non-woven fabric, hydrophilic foam, or a porous polymer matrix. The central active treatment zone is loaded with one or more active therapeutic ingredients effective to treat or alleviate tooth pain through mechanisms other than nerve anesthesia. Such active ingredients may include, without limitation: plant-derived extracts with documented anti-inflammatory or analgesic properties, anti-inflammatory factors, tissue repair-promoting agents, hemostatic agents, antimicrobial compounds for treating infectious etiology of dental pain, or other non-anesthetic therapeutic substances appropriate for oral mucosal drug delivery.

Upon contact with the moist environment of the oral cavity during use, the central active treatment zone is configured to release the loaded active ingredients in a sustained and controlled manner, enabling the therapeutic agents to penetrate the adjacent affected tissue at the site of application.

### C. Side Anchoring and Blocking Zones (Side Gauze—Fixing and Barrier Component)

The side anchoring and blocking zones are symmetrically disposed along the two longitudinal edges flanking the central active treatment zone, aligned with the short edges of the base support and isolation layer. The length of each side anchoring and blocking zone corresponds to the width of the base support and isolation layer, approximately 1.5 cm. The thickness of the side anchoring and blocking zones is substantially equal to that of the central active treatment zone, approximately 2.0 mm.

In the top view of FIG. 1, the surface of the side anchoring and blocking zones is shown to exhibit a hatched pattern, which is visually and structurally distinct from the grid pattern of the central active treatment zone, indicating a difference in material composition, surface treatment, or functional properties relative to the central active treatment zone.

The side anchoring and blocking zones perform a dual function of critical importance to the present invention.

First, Adhesion and Anchoring Function: The side anchoring and blocking zones are configured to provide the adhesive force necessary to firmly secure the entire patch structure to the moist gingival or tooth surface. Adhesion may be achieved by impregnating the side zones with a medical-grade pressure-sensitive adhesive, or by employing hydrogel materials that swell upon contact with saliva to generate viscous adhesion to the mucosal surface. This adhesion prevents the patch from slipping, shifting, or dislodging during the period of drug delivery, ensuring that the central active treatment zone remains stably aligned with and in contact with the targeted painful site.

Second, Drug Liquid Blocking Function: When the active ingredients loaded in the central active treatment zone dissolve and liquefy upon contact with the oral environment, the side anchoring and blocking zones act as physical containment barriers or absorption dams flanking the central zone. The side zones are configured to effectively intercept and block the liquefied or dissolved drug substance from flowing laterally outward to the sides of the patch or from being inadvertently swallowed by the patient. This confinement ensures that the dissolved active ingredients remain concentrated at the target treatment area, improving drug utilization efficiency, enhancing the localized therapeutic effect, and improving the user experience.

### DESCRIPTION OF THE WORKING PRINCIPLE

During use, the patient applies the side of the patch bearing the raised functional structures to the specific tooth or gingival area experiencing pain, with the planar base support and isolation layer facing outward toward the oral cavity.

Stage 1—Fixation and Containment: Upon application, the two side anchoring and blocking zones establish stable adhesive contact with the surrounding gingival or mucosal tissue, completing initial positioning and securing of the patch, while simultaneously forming a drug anti-spillage containment barrier on both lateral sides of the central active treatment zone.

Stage 2—Drug Release and Therapeutic Action: The slightly moist environment within the oral cavity permeates the central active treatment zone, activating and dissolving the active ingredients loaded therein. The dissolved active ingredients are released in a sustained manner and are intended to treat or resolve the underlying etiology of tooth pain.

Stage 3—Controlled Unidirectional Penetration: Aided by the external barrier effect of the base support and isolation layer on the outer surface of the patch, and the lateral blocking effect of the two side anchoring and blocking zones, the dissolved active ingredients are confined to the targeted treatment area and are directed to continuously and deeply penetrate into the closely adhering affected tissue, thereby maximizing therapeutic concentration at the site of pathology.

Stage 4—Pain Resolution: Following penetration of the active ingredients into the target tissue, the therapeutic agents exert their pharmacological or physical therapeutic mechanisms to address the underlying cause of tooth pain, providing causative treatment rather than mere symptomatic nerve anesthesia.

What is claimed is:

1. A multilayered toothache patch structure for targeted local oral administration, the patch comprising:
(a) a base support and isolation layer forming an outer surface of the patch, the base support and isolation layer being fabricated from a waterproof or semi-permeable polymeric material configured to substantially prevent permeation of saliva into the patch and to substantially prevent release of active ingredients from said outer surface;
(b) a central active treatment zone disposed on a first face of the base support and isolation layer, the central active treatment zone comprising a porous, biocompatible carrier material loaded with one or more active therapeutic ingredients for treating tooth pain; and
(c) at least two side anchoring and blocking zones disposed on the first face of the base support and isolation layer and symmetrically flanking the central active treatment zone along longitudinal edges thereof, the side anchoring and blocking zones being configured to provide adhesion to moist gingival or mucosal tissue and to act as a physical barrier preventing lateral migration of dissolved drug substance from the central active treatment zone.

2. The patch of claim 1, wherein the base support and isolation layer has a planar dimension of approximately 3.0 cm in length by 1.5 cm in width.

3. The patch of claim 1, wherein the central active treatment zone has a planar dimension of approximately 1.5 cm in length by 1.0 cm in width and a thickness of approximately 2.0 mm.

4. The patch of claim 1, wherein the central active treatment zone exhibits a grid or latticed surface texture configured to increase specific surface area and enhance active ingredient loading capacity.

5. The patch of claim 1, wherein the side anchoring and blocking zones exhibit a surface texture distinct from that of the central active treatment zone.

6. The patch of claim 1, wherein the side anchoring and blocking zones are impregnated with a medical-grade pressure-sensitive adhesive.

7. The patch of claim 1, wherein the side anchoring and blocking zones comprise a hydrogel material configured to swell upon contact with saliva to generate adhesive force to moist mucosal surfaces.

8. The patch of claim 1, wherein the carrier material of the central active treatment zone is selected from the group consisting of modified gauze, non-woven fabric, hydrophilic foam, porous polymer matrix, and combinations thereof.

9. The patch of claim 1, wherein the one or more active therapeutic ingredients comprise at least one of: a plant-derived extract having anti-inflammatory or analgesic properties, an anti-inflammatory factor, a tissue repair-promoting agent, a hemostatic agent, or an antimicrobial compound.

10. The patch of claim 1, wherein the patch is substantially rectangular in plan view.

11. The patch of claim 1, wherein the base support and isolation layer is selected from the group consisting of polyethylene film, polyurethane film, ethylene vinyl acetate copolymer film, polylactic acid film, and combinations thereof.

12. The patch of claim 1, wherein the central active treatment zone is configured to release the active therapeutic ingredients in a sustained and controlled manner upon contact with oral mucosal moisture.

13. A method of treating tooth pain in a subject, comprising: applying to an affected tooth or gingival area of the subject the multilayered toothache patch of claim 1 with the central active treatment zone and side anchoring and blocking zones in contact with the affected area; and maintaining the patch in contact with the affected area until a therapeutic amount of the active ingredient has been delivered to the tissue.
